(19) United States
(12) Patent Application Publication
(10) Pub. No.: US 2013/0090637 A1
(43) Pub. Date: Apr. 11, 2013

(54) CATHETER DEVICE AND METHOD FOR DENERVATION

(71) Applicant: John SLIWA, Los Altos Hills, CA (US)

(72) Inventor: John SLIWA, Los Altos Hills, CA (US)

(73) Assignee: ST. JUDE MEDICAL, INC., St. Paul, MN (US)

(21) Appl. No.: 13/253,219

(22) Filed: Oct. 5, 2011

(51) Int. Cl. A61B 18/18

(52) U.S. Cl. 606/14

(57) ABSTRACT

A method for denervation comprises: introducing a distal portion of a catheter to an interior of a vessel of patient, the catheter including an elongated catheter body extending longitudinally between a proximal end and a distal end along a longitudinal axis, the catheter body including the distal portion at the distal end and a catheter lumen from the proximal end to the distal end; delivering optical energy via the catheter lumen to the distal portion of the catheter body; emitting an optical beam outwardly from the distal portion, through an optical emission port disposed in the distal portion of the catheter body; and forming at least one trench using the emitted optical beam with sufficient intensity to a depth into a vessel wall of the vessel sufficient to cause tissue removal and physically sever at least one nerve associated with the vessel wall at the depth within that depth range.

### BACKGROUND OF THE INVENTION

The present invention relates generally to denervation, and more specifically to minimally invasive catheter devices and methods for physically severing one or more nerves in renal denervation or the like.

Hypertension (HTN), or high blood pressure (HBP), is defined as a consistently elevated blood pressure (BP) greater than or equal to 140 mmHg systolic blood pressure (SBP) and 90 mmHg diastolic blood pressure (DBP). Hypertension is a “silent killer” that is not associated with any symptoms and in 95% of cases (primary hypertension) the specific cause is unknown. In the remaining 5% of patients (secondary hypertension), specific causes including chronic renal disease, diseases of the adrenal gland, coarctation of the aorta, thyroid dysfunction, alcohol addiction, pregnancy or the use of birth control pills are present. In secondary hypertension, when the root cause is treated, blood pressure usually returns to normal.

It is generally accepted that the causes of hypertension are multi-factorial, with a significant factor being the chronic hyper-activation of the sympathetic nervous system (SNS), especially the renal sympathetic nerves. Renal sympathetic efferent and afferent nerves, which lie in the wall of the renal artery, have been recognized as a critical factor in the initiation and maintenance of systemic hypertension. Renal arteries, like all major blood vessels, are innervated by perivascular sympathetic nerves that traverse the length of the arteries. The perivascular nerves consist of a network of axons, terminals, and varicosities, which are distributed mostly in the medial-adventitial and adventitial layers of the arterial wall.

Signals coming in to the kidney travel along efferent nerve pathways and influence renal blood flow, trigger fluid retention, and activate the renin-angiotensin-aldosterone system cascade. Renin is a precursor to the production of angiotensin II, which is a potent vasoconstrictor, while aldosterone regulates how the kidneys process and retain sodium. All of these mechanisms serve to increase blood pressure. Signals coming out of the kidney travel along afferent nerve pathways integrated within the central nervous system, and lead to increased systemic sympathetic nerve activation. Chronic over-activation can result in vascular and myocardial hypertrophy and insulin resistance, causing heart failure and kidney disease.

Previous clinical studies have documented that denervating the kidney has a positive effect for both hypertension and heart failure patients. Journal articles published as early as 1936 review surgical procedures called either sympathectomy or splanchnicectomy, to treat severe hypertension. A 1953 JAMA article by Smithwick et al. presented the results of 1,266 cases of surgical denervation to treat hypertension. The results included radiographic evidence of hearts that had remodeled after the surgery, while also showing significant blood pressure declines. Additional articles published in 1955 and 1964 demonstrated that the concept of using renal denervation to lower blood pressure and treat heart failure was viable. However, given the highly invasive and traumatic nature of the procedure and the advent of more effective antihypertensive agents, the procedure was not widely employed.

More recently, partly as a result of developments in the 1990's in catheter technology, catheter ablation has been used for renal sympathetic denervation. Renal denervation is a method whereby amplified sympathetic activities are suppressed by heat injury to nerve fibers to treat hypertension or other cardiovascular disorders and chronic renal diseases. The objective of renal denervation is to neutralize the effect of renal sympathetic system which is involved in arterial hypertension. The renal sympathetic efferent and afferent nerves lie within and immediately adjacent to the wall of the renal artery. Energy is delivered via a catheter to ablate the renal nerves in the right and left renal arteries in order to disrupt the chronic activation process. As expected, early results confirm the important role of renal sympathetic nerves in resistant hypertension and establish that renal sympathetic denervation is of therapeutic benefit in this patient population. In clinical studies, therapeutic renal sympathetic denervation has produced predictable, significant, and sustained reductions in blood pressure in patients with resistant hypertension.

Catheters are flexible, tubular devices that are widely used by physicians performing medical procedures to gain access into interior regions of the body. A catheter can be used for ablating renal sympathetic nerves in therapeutic renal sympathetic denervation to achieve reductions of blood pressure in patients suffering from renal sympathetic hyperactivity associated with hypertension and its progression.

### BRIEF SUMMARY OF THE INVENTION

Embodiments of the present invention provide minimally invasive catheter devices and methods for denervation that involve physically severing one or more nerves using a tool provided in a distal portion of the catheter that is introduced into a vessel of a patient such as a renal artery or vein. In some preferred embodiments, the denervation does not substantially raise the temperature of the region of the vessel being denervated. In specific embodiments, the one or more nerves being physically severed are cauterized to stop any potential or actual bleeding or leakage, preferably using a tool provided in the distal portion of the catheter.

One exemplary embodiment of the invention is directed to a method for achieving denervation of nerves running generally along the length of a vessel or bodily conduit with superior efficacy and/or minimal concomitant tissue damage. The denervation is performed using a catheter with a distal treatment end, an intermediate extended flexible lumen body, and a proximal control-handle, motion controller, or grip. A tissue-severing tool is provided at the distal treatment end which is operative to physically sever at least some nerve-related tissue juxtaposed to the distal treatment end and situated at a depth into the juxtaposed tissue. The operator obtains knowledge of where the target nerves either actually are or where they might be in terms of angular position or position range about the vessel axis and/or with regard to actual or likely depth. The operator inserts the catheter into a patient's body and directs or operates the distal tool to form one or more trenches into the juxtaposed tissue to a depth sufficient to sever substantially all underlying target nerve fibers which fall within the length of each such trench. The procedure forms one or more such trenches which cumulatively trench and sever substantially all known or possible nerves at the known angle(s) and/or at the anticipated depth(s). The nerves run generally in the longitudinal direction of the vessel at certain angular positions along the circumference and at certain depths from the interior surface of the vessel wall. These angular positions may be known or estimated if unknown. The angular positions, as of today, are unknown. The depth range is estimated based on historical patient populations having different lumen diameters and lumen wall thicknesses, for example. The trenches each have a trench width narrower than a trench length and a trench depth larger than the trench width, the width being that occurring during trench formation. The trenching physically severs nerves at one or more known or unknown angles or within the trenched depth zone with a minimum of total tissue target volume destroyed. The method thereby offers one or both of superior efficacy due to an assured approximate 100 percent severance of possible or known nerve targets, and the least possible cumulative tissue destruction while still severing all or most intended nerves.

In accordance with an aspect of the present invention, a method for denervation comprises: introducing a distal portion of a catheter to an interior of a vessel of a patient, the catheter including an elongated catheter body extending longitudinally between a proximal end and a distal end along a longitudinal axis, the catheter body including the distal portion at the distal end and a catheter lumen from the proximal end to the distal end; delivering optical energy via the catheter lumen to the distal portion of the catheter body; emitting an optical beam outwardly from the distal portion, through an optical emission port disposed in the distal portion of the catheter body; and forming at least one trench using the emitted optical beam with sufficient intensity to a depth into a vessel wall of the vessel sufficient to cause tissue removal and physically sever at least one nerve associated with the vessel wall at the depth.

In some embodiments, the optical beam is emitted to produce severing actions that each sever a portion of the at least one nerve at a time and that cumulatively sever the at least one nerve entirely. The method further comprises cauterizing remaining tissue in the vicinity of the at least one nerve being physically severed, by directing from the distal portion cauterizing energy to the remaining tissue. The cauterizing is performed by directing an optical beam from the optical emission port to the tissue in the vicinity of the at least one severed nerve. Forming the at least one trench to physically sever the at least one nerve and the cauterizing of the remaining tissue in the vicinity of the at least one nerve being physically severed are performed substantially sequentially or in a time-interleaved manner. The method further comprises flushing blood away from a region adjacent or within the at least one trench with a flushant such as saline emanating from the distal portion.

In specific embodiments, the method further comprises physically advancing the optical emission port toward or into the vessel wall before or during forming the at least one trench using the emitted optical beam. The vessel extends longitudinally in a longitudinal direction and the vessel wall is formed circumferentially around the interior of the vessel. The at least one trench, when projected longitudinally on a plane normal to the longitudinal direction of the vessel, preferably extends around a complete circumferential loop. The at least one trench may be substantially lateral to the longitudinal direction of the vessel. The at least one trench may comprise a helical trench. The optical beam used in forming the at least one trench has sufficiently low average power so as to avoid substantially raising a temperature of a region of the vessel wall receiving the emitted optical beam. The optical energy is in an ultraviolet wavelength range. The optical energy, if in the ultraviolet range, is inherently nonheating and if instead in a nonultraviolet range may be made nonheating in the widely known manner of using sufficiently low average power.

In accordance with another aspect of this invention, a method for denervation comprises: introducing a distal portion of a catheter to an interior of a vessel of a patient, the catheter including an elongated catheter body extending longitudinally between a proximal end and a distal end along a longitudinal axis, the catheter body including the distal portion at the distal end and a catheter lumen from the proximal end to the distal end; and operating a tool disposed in the distal portion of the catheter to form at least one trench with physical tissue separation in a vessel wall of the vessel and physically sever at least one nerve associated with the vessel using the tool.

In some embodiments, the physical tissue separation of the at least one trench is temporary. The method further comprises cauterizing remaining tissue in the vicinity of the at least one nerve being physically severed, by directing from the distal portion cauterizing energy or chemical to the remaining tissue. The cauterizing is performed by one of: (i) directing an optical beam from an optical emission port in the distal portion to the remaining tissue in the vicinity of the at least one nerve being physically severed; (ii) placing a heated severing tool on the remaining tissue in the vicinity of the at least one nerve being physically severed; (iii) delivering RF energy to heat the remaining tissue in the vicinity of the at least one nerve being physically severed; (iv) delivering heat from a heating element in the distal portion toward the remaining tissue in the vicinity of the at least one nerve being physically severed; (v) emanating a hot fluid from the distal portion toward the remaining tissue in the vicinity of the at least one nerve being physically severed; and (vi) delivering a chemical cauterizing agent to the remaining tissue in the vicinity of the at least one nerve being physically severed. The physical severing of the at least one nerve and the cauterizing of the remaining tissue in the vicinity of the at least one nerve being physically severed are performed substantially sequentially or in a time-interleaved manner.

In specific embodiments, the physical severing of the at least one nerve is performed by one of (i) delivering optical energy via the catheter lumen to the distal portion of the catheter body, emitting an optical beam outwardly from the distal portion, through an optical emission port disposed in the distal portion of the catheter body, and forming the at least one trench using the emitted optical beam with sufficient intensity to a depth into the vessel wall of the vessel of the patient to cause tissue removal and physically sever the at least one nerve; or (ii) advancing a penetrating severing tool into the vessel wall with physical tissue separation to physically sever the at least one nerve in the vessel.

In some embodiments, forming the at least one trench is performed without substantially raising a temperature of a region of the vessel wall in the vicinity of the at least one trench. The method further comprises cauterizing remaining tissue in the vicinity of the at least one nerve being physically severed, by delivering a chemical cauterizing agent to the remaining tissue in the vicinity of the at least one nerve being physically severed without substantially raising a temperature of a region of the vessel wall in the vicinity of the at least one trench. The method further comprises combining penetrating severing motions and circumferential motions such as to move upon the interior lumen surface to the next trench portion to be penetration-cut or to slice along a circumferential direction during a penetrated condition.

In specific embodiments, the vessel extends longitudinally in a longitudinal direction and the vessel wall is formed circumferentially around the interior of the vessel. The at least one trench, when projected longitudinally on a plane normal to the longitudinal direction of the vessel, extends around a complete circumferential loop. Each of the at least one trench has a lateral length which is greater than a width and a depth into the vessel wall which is greater than the width, the lateral length being substantially perpendicular to the longitudinal direction of the vessel. Each of the at least one trench has a surface length along an interior surface of the vessel wall and an at-depth length at a trench bottom of the trench at the depth into the vessel wall, the at-depth length being greater than the surface length of the trench. The at least one trench may comprise a helical trench.

In some embodiments, the tool comprises a pair of diametrically opposed penetrating severing tools, and the penetrating severing tools are operated to cut simultaneously dual diametrically opposed trenches at least partially through the vessel wall. The tool may comprise a penetrating severing tool, and operating the tool comprises translating the penetrating severing tool while advancing the penetrating severing tool into the vessel wall to form the at least one trench in the vessel wall.

In specific embodiments, the tool comprises optical energy delivered via the catheter lumen to the distal portion of the catheter body, to emit an optical beam outwardly from the distal portion, through an optical emission port disposed in the distal portion of the catheter body, so as to form the at least one trench using the emitted optical beam with sufficient intensity to a depth into the vessel wall of the vessel of the patient to cause tissue removal and physically sever the at least one nerve. Operating the tool includes maintaining an optical path for the emitted optical beam to a trench bottom of the at least one trench. The method further comprises directing a fluid from the distal portion to flush the at least one trench.

In some embodiments, the tool is operated to form a plurality of trench portions along a trenching path with webs of connecting tissue left between the trench portions along the trenching path, the webs of connecting tissue being any of: (a) untrenched portions of the lumen wall, (b) portions of the lumen wall which are trenched to a lesser depth than their immediately adjacent trench portions, or (c) portions of the lumen wall which are trenched only beneath the interior lumen surface thereby leaving bridging webs of tissue at the lumen interior surface. The webs of connecting tissue do not reach a depth of the at least one nerve to be physically severed.

In accordance with another aspect of the present invention, a catheter apparatus for denervation comprises: an elongated catheter body extending longitudinally between a proximal end and a distal end along a longitudinal axis, the catheter body including a distal portion at the distal end and a catheter lumen from the proximal end to the distal end; an optical emission port disposed in the distal portion of the catheter body to emit an optical beam outwardly from the distal portion; and an optical energy delivery conduit extending through the catheter lumen to the optical emission port to deliver optical energy to the optical emission port to produce the emitted optical beam, the optical emission port capable of delivering the emitted optical beam with sufficient intensity to a depth into a vessel wall of a vessel of a patient sufficient to cause tissue removal and physically sever at least one nerve associated with the vessel wall at the depth. The optical emission port is oriented substantially lateral to the longitudinal axis.

In some embodiments, the optical beam is emitted to produce severing actions that each sever a portion of the at least one nerve at a time and that cumulatively sever the at least one nerve entirely. The catheter apparatus further comprises a cauterizing member in the distal portion, the cauterizing member comprising one of: (i) the optical emission port to direct an optical beam to heat remaining tissue in the vicinity of the at least one nerve being physically severed; (ii) an RF member to deliver RF energy to heat remaining tissue in the vicinity of the at least one nerve being physically severed; (iii) a heating element to deliver heat to remaining tissue in the vicinity of the at least one nerve being physically severed; (iv) a hot fluid emanated from the distal portion toward remaining tissue in the vicinity of the at least one nerve being physically severed; or (v) a delivery orifice to deliver a chemical cauterizing agent to remaining tissue in the vicinity of the at least one nerve being physically severed.

In specific embodiments, the catheter apparatus further comprises an optical beam redirector to redirect the optical energy from the optical energy delivery conduit to the optical emission port in a direction substantially lateral to the longitudinal axis. The optical energy is selected from the group consisting of laser energy and LED energy. The optical energy is in an ultraviolet wavelength range. The optical beam is emitted with sufficiently low average power so as to avoid substantially raising the temperature of the region of the vessel wall receiving the emitted optical beam. The catheter apparatus further comprises a control member to control the emitted optical beam to form a severing trench in the vessel wall, the trench having a surface length measured along an interior surface of the vessel wall. In any event the optical beam, when severing, is either in the ultraviolet range or is in a nonultraviolet range with a sufficiently low average power so as to avoid substantially raising the temperature of the region of the vessel wall receiving the emitted optical beam. Again, sufficiently long ultraviolet pulses of high average power can be made to heat (cauterize) albeit inefficiently compared to other non-UV wavelengths.

In accordance with another aspect of this invention, a catheter apparatus for denervation comprises: an elongated catheter body extending longitudinally between a proximal end and a distal end along a longitudinal axis, the catheter body including a distal portion at the distal end and a catheter lumen from the proximal end to the distal end; and a tool disposed in the distal portion of the catheter to form a trench with physical tissue separation in a vessel wall of a vessel of a patient and physically sever at least one nerve associated with the vessel using the tool.

In some embodiments, the physical tissue separation of the trench is temporary. The catheter apparatus further comprises a cauterizing member in the distal portion, the cauterizing member comprising one of: (i) an optical emission port to direct an optical beam to heat remaining tissue in the vicinity of the at least one nerve being physically severed; (ii) a heated severing tool to be thermally coupled to remaining tissue in the vicinity of the at least one nerve being physically severed; (iii) an RF member to deliver RF energy to heat remaining tissue in the vicinity of the at least one nerve being physically severed; (iv) a heating element to deliver heat to remaining tissue in the vicinity of the at least one nerve being physically severed; (v) a hot fluid emanated from the distal portion toward remaining tissue in the vicinity of the at least one nerve being physically severed; or (vi) a delivery orifice to deliver a chemical cauterizing agent to remaining tissue in the vicinity of the at least one nerve being physically severed

In specific embodiments, the tool comprises an optical emission port disposed in the distal portion of the catheter body to emit an optical beam outwardly from the distal portion, and an optical energy delivery conduit extending through the catheter lumen to the optical emission port to deliver optical energy to the optical emission port. The optical emission port is oriented substantially lateral to the longitudinal axis. The catheter apparatus further comprises an optical beam redirector to redirect the optical energy from the optical energy delivery conduit to the optical emission port in a direction substantially lateral to the longitudinal axis. The optical energy is selected from the group consisting of laser energy and LED energy. The optical energy is in an ultraviolet wavelength range. The optical beam, regardless of wavelength, is emitted with sufficiently low average power so as to avoid substantially raising the temperature of the region of the vessel wall receiving the emitted optical beam.

In some embodiments, the tool comprises a penetrating severing tool, and the catheter apparatus further comprises at least one control member configured to perform at least one of: translating the penetrating severing tool to or along a vessel wall location before severing at the vessel wall location; translating the penetrating severing tool along the vessel wall while the penetrating severing tool is severing, the severing including one or more of inward depth cutting or translational lateral cutting to achieve a severed lateral length; causing a puncturing and substantially nonslicing cutting tip of the penetrating severing tool to penetrate the tissue without substantial lateral translation along the vessel wall; or causing the penetrating severing tool to puncture tissue in a substantially nonslicing but still penetrating manner. In specific embodiments, the tool comprises a pair of diametrically opposed penetrating severing tools, and the catheter apparatus further comprises a control member to operate the penetrating severing tool to cut simultaneously dual diametrically opposed trenches in the vessel wall.

In some embodiments, the catheter body has a size between about 4 French and about 8 French. The catheter has dimensions and flexural properties so as to be deliverable into at least one of a renal artery or a renal vein. The catheter apparatus further comprises a flush member in the distal portion to direct a fluid to flush the trench.

In accordance with another aspect of the invention, a catheter apparatus for denervation comprises: an elongated catheter body extending longitudinally between a proximal end and a distal end along a longitudinal axis, the catheter body including a distal portion at the distal end and a catheter lumen from the proximal end to the distal end; and means disposed in the distal portion of the catheter for forming a trench with physical tissue separation in a vessel wall of a vessel of a patient and physically severing at least one nerve associated with the vessel.

These and other features and advantages of the present invention will become apparent to those of ordinary skill in the art in view of the following detailed description of the specific embodiments.

### BRIEF DESCRIPTION OF THE DRAWINGS

FIG. 1 shows a catheter device disposed in a vessel of a patient for denervation using optical energy according to one embodiment of the invention.

FIG. 1A is a schematic view showing additional and alternative features of the distal portion of the catheter device of FIG. 1.

FIG. 2 is a schematic perspective view illustrating trenches on a vessel.

FIG. 3 illustrates multiple trenches that span open arc segments around the longitudinal axis of the vessel of the patient.

FIG. 4 illustrates the multiple trenches of FIG. 3 that, when projected longitudinally onto any lateral plane which is perpendicular to the longitudinal axis, span a closed loop around the longitudinal axis of the vessel.

FIG. 5 illustrates a single helical trench that, when projected longitudinally onto any lateral plane which is perpendicular to the longitudinal axis, spans a closed loop around the longitudinal axis of the vessel.

FIG. 6 shows a catheter disposed in a vessel of a patient for denervation using a severing tool according to another embodiment of the invention.

FIG. 7 shows a catheter disposed in a vessel of a patient for denervation using a severing tool according to another embodiment of the invention.

FIG. 8 is a lateral cross-sectional view of a vessel of a patient showing an example of a trench that is comprised of trench portions with webs of connecting tissue therebetween.

### DETAILED DESCRIPTION OF THE INVENTION

In the following detailed description of the invention, reference is made to the accompanying drawings which form a part of the disclosure, and in which are shown by way of illustration, and not of limitation, exemplary embodiments by which the invention may be practiced. In the drawings, like numerals describe substantially similar components throughout the several views. Further, it should be noted that while the detailed description provides various exemplary embodiments, as described below and as illustrated in the drawings, the present invention is not limited to the embodiments described and illustrated herein, but can extend to other embodiments, as would be known or as would become known to those skilled in the art. Reference in the specification to “one embodiment,” “this embodiment,” or “these embodiments” means that a particular feature, structure, or characteristic described in connection with the embodiment is included in at least one embodiment of the invention, and the appearances of these phrases in various places in the specification are not necessarily all referring to the same embodiment. Additionally, in the following detailed description, numerous specific details are set forth in order to provide a thorough understanding of the present invention. However, it will be apparent to one of ordinary skill in the art that these specific details may not all be needed to practice the present invention. In other circumstances, well-known structures, materials, circuits, processes and interfaces have not been described in detail, and/or may be illustrated in block diagram form, so as to not unnecessarily obscure the present invention.

In the following description, relative orientation and placement terminology, such as the terms horizontal, vertical, left, right, top and bottom, is used. It will be appreciated that these terms refer to relative directions and placement in a two dimensional layout with respect to a given orientation of the layout. For a different orientation of the layout, different relative orientation and placement terms may be used to describe the same objects or operations.

Exemplary embodiments of the invention, as will be described in greater detail below, provide minimally invasive catheter devices and methods for denervation that involve physically severing one or more nerves using a tool provided in a distal portion of the catheter that is introduced into a vessel of a patient such as a renal artery or vein.

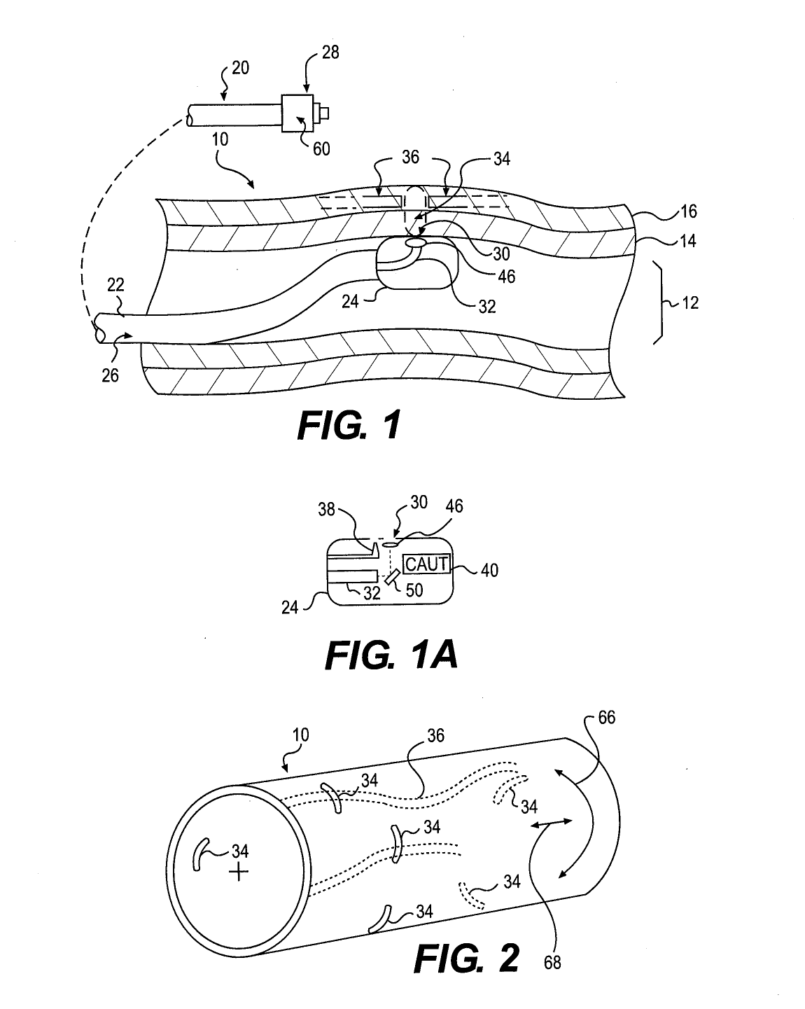

FIG. 1 shows a catheter device disposed in a vessel of a patient for denervation using optical energy according to one embodiment of the invention. The vessel 10 has a vessel wall that defines a lumen 12 such as a blood lumen. In the specific embodiment shown, the vessel wall includes a muscle layer 14 and a nerve layer 16. The catheter 20 has an elongated catheter body 22 extending longitudinally between a proximal end and a distal end along a longitudinal axis. The catheter body 22 includes a distal portion 24 at the distal end, a catheter lumen 26 from the proximal end to the distal end, and typically a handle 28 at the proximal end to manipulate or operate the catheter body 22 and/or other components such as denervation tools, sensors, and the like. The catheter body 22 typically has a size between about 4 French and about 8 French. The catheter preferably has dimensions and flexural properties so as to be deliverable into a renal artery or vein. The catheter 20 may be introduced into the lumen 12 of the vessel 10 using a guidance sheath or guiding wire (neither shown), or the like.

In FIG. 1, the denervation tool employs optical energy. An optical emission port 30 is disposed in the distal portion 24 of the catheter body 22 to emit an optical beam outwardly from the distal portion 24. An optical energy delivery conduit 32 extends through the catheter lumen 26 to the optical emission port 30 to deliver optical energy to the optical emission port 30 to produce the emitted optical beam. The optical emission port 30 is capable of delivering the emitted optical beam with sufficient intensity to a depth into the vessel wall of the vessel 10 sufficient to cause tissue removal and physically sever at least one nerve associated with the vessel wall at the depth within that depth range. The optical emission port 30 may be placed in a space which is flushed with blood, such as a blood lumen 12. The tissue removal produces a trench 34 in the vessel wall. The trench 34 has a surface length measured along an interior surface of the vessel wall. The trench 34 may be formed in various ways, including a series or array of holes that are formed sequentially or simultaneously. The trench 34 presents physical tissue separation in the vessel wall and the physical tissue separation of the trench 34 lasts at least for some amount of time, so that the separation can be temporary or permanent. The nerve may be in the vessel wall or juxtaposed to the vessel wall. In the example shown, the emitted optical beam causes tissue removal through the muscle layer 14 and at least partly through the nerve layer 16 to physically sever a nerve 36. It is preferable to maintain an optical path for the emitted optical beam to a trench bottom of the trench 34, for instance, by flushing the trench 34, as with saline, to assure optical penetration to the current trench bottom. The saline or other delivered irrigant may also usefully mechanically temporarily bias the trench walls to spread apart somewhat more. These both ensure that the optical beam will reach the nerve 36 and that a safe path exists for microexplosive or outdiffusing byproducts to leave the denervation area. A water (e.g., saline) tip flush 38 may be provided to flush the trench 34 and beam path using water or some other fluid, as seen in FIG. 1A. The optical emission port 30 is oriented substantially lateral to the longitudinal axis (i.e., preferably within about 30 degrees of being perpendicular, more preferably within about 20 degrees, and most preferably within about 10 degrees). A few degrees of tilt (e.g., 10 degrees) to the lumen wall may help reduce reflections. Before or during forming the trench using the emitted optical beam, the operator may physically advance the optical emission port 30 toward or into the vessel wall such as to reduce working distance, position a focus, or displace blood in the optical path.

The nerve severing action may be of a continuous nature or an incremental nature. For incremental severing, the optical beam is emitted to produce severing actions that each sever a portion of the nerve at a time and that cumulatively sever the nerve entirely. In addition, the exposed nerve ends, having been severed, are preferably cauterized or necrosed to stop any potential bleeding or leakage of fluids inward or outward. The same may apply to any small severed blood vessels in the severed region. Cauterizing may be performed by directing an optical beam via the optical emission port 30 to heat remaining tissue in the vicinity of the nerve 36 being physically severed. Other ways of cauterization may be performed using a different cauterizing member 40 in the distal portion 24 to direct cauterizing energy to the remaining tissue (FIG. 1A). Examples of such a cauterizing member 40 include: an RF member to deliver RF energy to heat the remaining tissue, a heating element to deliver heat to the remaining tissue (by emanating, radiating, generating, or conducting heat), a hot fluid emanated from the distal portion 24 toward the remaining tissue, and a delivery orifice in the distal portion 24 to deliver a chemical cauterizing agent (preferably unheated chemical cauterizing, including but not limited to carbolic acid, phenol, trichloroacetic acid, and other strong acids or alkali) to the remaining tissue in the vicinity of the nerve being physically severed (delivering a chemical cauterizing agent can be considered directing a chemical cauterizing energy to the remaining tissue). Forming the trench 34 to physically sever the nerve 36 and cauterizing of the remaining tissue in the vicinity of the nerve 36 being physically severed may be performed substantially simultaneously. In some cases, the incremental nerve severing action and the cauterization can be interleaved. Trenching uniquely allows for at-depth cauterization, especially chemical cauterization which would otherwise be impossible at depth.

The nerve severing action using the severing tool is always the first to break the nerve connection of a nerve to be physically severed (i.e., not the cauterizing). Denervation is achieved by physically severing one or more nerves and not by ablation. In addition to preventing bleeding or leakage, cauterization may help to seal the trench 34 and ensure that the severed ends of the nerve do not reconnect. Cauterization may toughen the tissue at the location of the nerve being severed to prevent tearing, particularly at the ends of the trenches.

An optical lens 46 may be provided to focus the optical beam into the vessel wall. The optical energy delivery conduit 32 may include one or more optical fibers. The optical fiber(s) may be bent or include a curved portion from the longitudinal direction to the lateral direction to deliver optical energy to the optical emission port 30, as seen in FIG. 1. Alternatively, an optical beam redirector 50 is provided to redirect the optical energy from the optical energy delivery conduit 32 to the optical emission port 30 in a direction substantially lateral to the longitudinal axis of the catheter body 22, as seen in FIG. 1A. Examples of the redirector 50 include an optical mirror, reflector, refractor, or prism. The optical redirector 50 may have a reflective coating optimized for the wavelength of the optical energy.

The optical energy may be laser energy, LED energy, or the like. In some embodiments, the optical energy is in the ultraviolet wavelength range. An example is optical energy produced by an Excimer laser. Optical energy in the ultraviolet wavelength range inherently does not cause heating of the target tissue (or is at least substantially nonheating). If the optical energy causes heating, the optical beam is preferably emitted with sufficiently low average power (e.g., low duty cycle or short pulses) so as to avoid substantially raising the temperature of the region of the vessel wall receiving the emitted optical beam (e.g., temperature rise above bodily temperature of 37 Deg C. (Centigrade) of preferably less than about 8 Deg C. (up to about 45 Deg C.), more preferably less than about 4 Deg C. (up to about 41 Deg C.), most preferably less than about 2 deg C. (up to about 39 Deg C.)). For example, the optical beam is delivered at short pulses on the order of 10⁻¹⁵ second with periods on the order of 10⁻¹² second between pulses. It is a simple known exercise for a developer of a particular optical emitter to test treated tissue target sites on the bench and determine for specific conditions of wavelength, pulse length, pulse repetition frequency, focus (if any), tissue blood perfusion and cooling, and cooling due to saline flushant, how much the tissue is heated.

A control member 60 is provided to control the emitted optical beam to form the trench 34 in the vessel wall. The control member 60 may be provided at or near the proximal end of the catheter 20. One part of the control member 60 may be used to control the optical energy delivered to the optical emission port 30. Another part may be used to control the placement and movement of the optical emission port 30 by manipulating the catheter body 22 and the distal portion 24. The control member 60 may produce manual rotation of the whole catheter 20, autorotation, selective manual tilting of a mirror/prism, selective auto tilting of a mirror/prism, etc. One preferred automated approach has auto rotate around a rotation angle and auto translate along the vessel length of the vessel 10. The control member 60 causes the emitted optical beam to be steered, directed, or focused to form an extended trench 34, progressively, incrementally, continuously, or otherwise. In some preferred embodiments, the extended trench has a length measured at the interior surface of the vessel wall that is shorter than the length measured at the trench bottom, thereby reducing interior-surface damage. Extended trench based severing allows for superior denervation efficacy relative to thermal ablation denervation alone because the denervation practitioner can statistically cut more target nerves with less total vessel damage or stenosis.

FIG. 2 is a schematic perspective view illustrating multiple trenches 34 on a vessel 10. The vessel 10 extends longitudinally in a longitudinal direction and the vessel wall is formed circumferentially around the interior of the vessel 10. One or more trenches 34 formed are preferably substantially lateral to the longitudinal direction of the vessel 10 or along circumferential paths (i.e., preferably within about ±10 degrees of being perpendicular to longitudinal, more preferably within about ±7 degrees, and most preferably within about ±5 degrees). The vessel 10, being pressurized with blood, is subjected to hoop stresses in the hoop direction 66 (tensile stresses along the circumference) as opposed to the axial direction 68. Lateral (near or at circumferential) trenching minimizes tearing hoop wall stress components which might otherwise pull the trench open if the trench were oriented more longitudinally rather than laterally. The trenches 34 may be curvilinear, circumferential (shown), arc-shaped, or helical. A helical trench will have a tight pitch so that the trench is substantially lateral with respect to the longitudinal direction of the vessel 10. The one or more trenches 34, when projected longitudinally on a plane normal to the longitudinal direction of the vessel 10, may overlap and preferably extend cumulatively around a substantially complete circumferential 360 degrees. This would assure severing of any nerve running along the vessel 10 at any clock position.

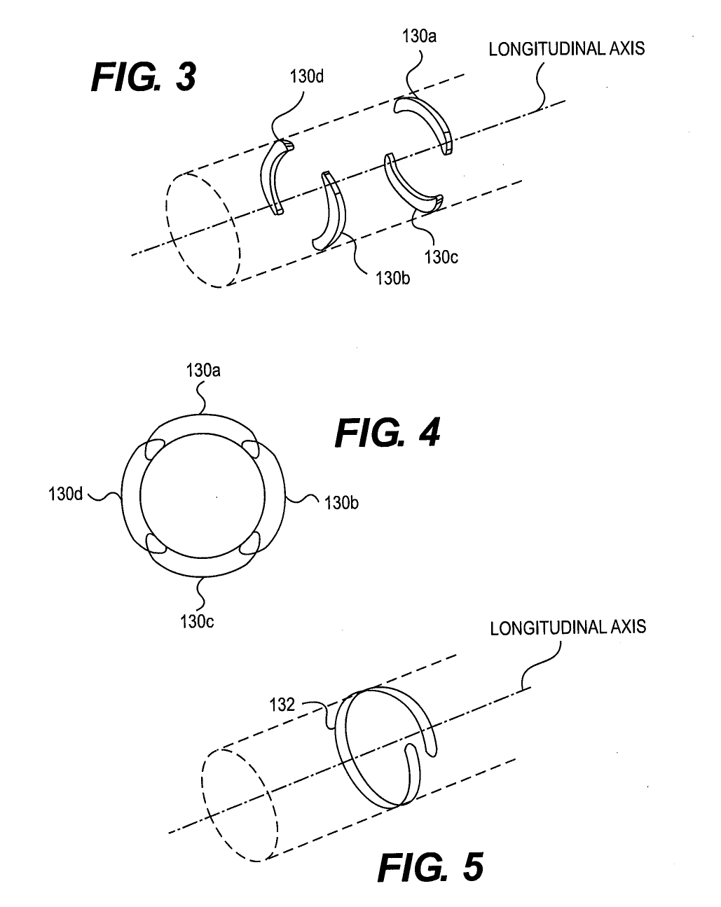

FIG. 3 illustrates multiple trenches that span open arc segments 130 (130a, 130b, 130c, 130d) around the longitudinal axis of the vessel of the patient. The open arc segments 130 are distributed in a staggered configuration. FIG. 4 illustrates the open arc segments 130 of FIG. 3 that, when projected longitudinally onto any lateral plane which is perpendicular to the longitudinal axis, span a closed loop (i.e., a complete circumferential loop) around the longitudinal axis of the vessel. It is noted that a single helical trench 132, when projected longitudinally onto any lateral plane, can also span a closed loop around the longitudinal axis of the vessel (see FIG. 5).

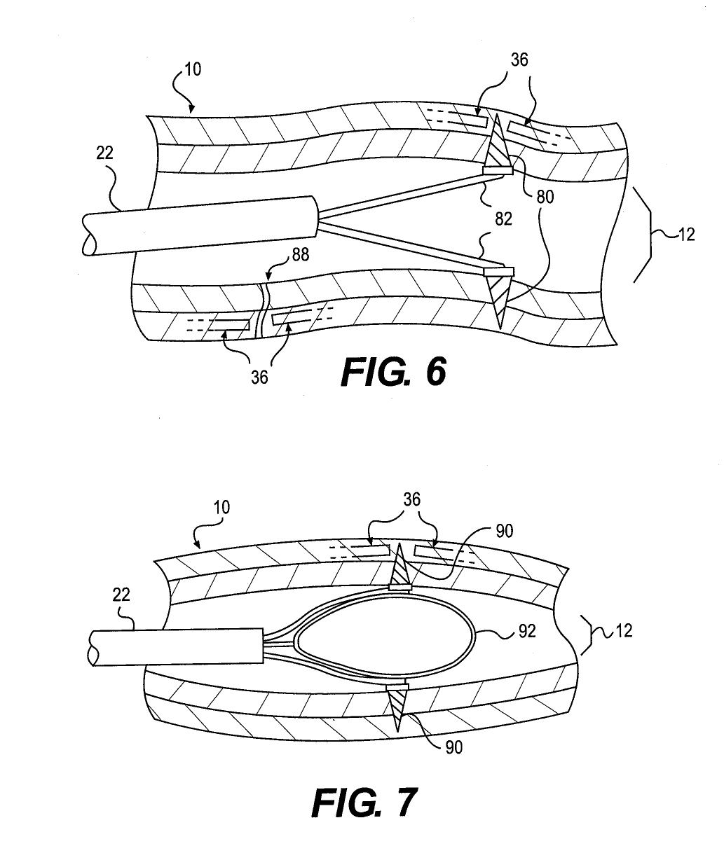

FIG. 6 shows a catheter disposed in a vessel of a patient for denervation using a severing tool according to another embodiment of the invention. In this embodiment, the tool for forming one or more trenches or perforations 34 in the vessel 10 is a penetrating severing tool 80, which is advanced into the vessel wall causing physical tissue separation to physically sever at least one nerve 36 associated with the vessel 10. Examples of the severing tool 80 include a slicing blade, a cleaving blade, a puncturing (nonslicing) blade, and other puncturing cutting implements. A control member 60 is used to translate or vibrate the penetrating severing tool 80 before severing at that location, translating the penetrating severing tool 80 along the wall while the severing tool is severing (the severing including inward depth cutting and/or translational lateral cutting to achieve a severed lateral length), translating the penetrating severing tool to a wall location before severing at that location, causing a puncturing and substantially nonslicing cutting tip of the penetrating severing tool 80 to penetrate the tissue without substantial lateral translation along the vessel wall, or causing the penetrating severing tool 80 to puncture tissue in a substantially nonslicing but still penetrating manner. The puncture may take various shapes, including round, square, rectangular, and narrow slit. The severing tool 80 may be used as a cauterizing tool by heating the severing tool 80 and placing it on or otherwise thermally coupling it to the remaining tissue in the vicinity of the nerve 36 being physically severed.

The specific embodiment of FIG. 6 has a pair of diametrically opposed penetrating severing tools 80 supported by a pair of arms 82. The arms 82 may be resiliently biased outwardly. For example, the arms 82 are constrained inside the catheter body 22 and, after placement at a target location inside the vessel 10, the arms 82 are pushed forward distally and then triggered or allowed to push or snap the severing tools 80 outwardly toward or into the tissue wall. The diametrically opposed configuration creates a load balance. The arms 82 are made of Nitinol or a similar material. The control member 60 is used to manipulate or operate the penetrating severing tool 80 to cut simultaneously dual diametrically opposed trenches or punctures in the vessel wall. FIG. 6 further shows cauterization 88 of remaining tissue associated with a severed nerve 34. Severing tools 80 might instead cut via a momentary snap-outward action while having a normal undeployed parked position away from the lumen walls or upon the interior lumen wall.

FIG. 7 shows a catheter disposed in a vessel of a patient for denervation using a severing tool according to another embodiment of the invention. This embodiment shows a pair of diametrically opposed penetrating severing tools 90 similar to the severing tools 80 of FIG. 6. This embodiment employs a preferably saline inflatable balloon 92 which is deployed distally from the distal end of the catheter 20 and inflated after placement of the tools 90 at a target location inside the vessel 10. Some advantages of the device of FIG. 7 over that of FIG. 6 are that the balloon can serve a few helpful functions such as providing any needed level of severing force, providing a fluoroscopy-opaque filling event if a contrast agent inflation is employed, and providing a degree of immobilization of the severing tools 90 along the long dimension of the lumen for increased safety.

According to embodiments of this invention, a trench has a width and a length measured on the interior surface of the vessel wall and a depth measured into the wall. Typically the length will be larger than the width, such as 2 to 200 or more times larger. The depth is typically larger than the width in order to reach and sever the underlying nerve which may be at a depth of 2-4 or so millimeters as is known, for example. It is important to note that functional nerve severance occurs once the trench is formed even if the trench naturally recloses upon itself after the severing action stops. Thus, we emphasize that by trench width we mean the width before any such closure may occur. The trench width for an optical beam used to form the trench will typically be roughly the optical beam diameter, whereas the width for a penetrating severing blade or punch will typically be the thickness of the blade or the diameter/width of the punch. Severing action typically comprises tissue-inward (radially outward) depth-wise cutting; however, in some cases, one can also translate a cutting tool which has already penetrated tissue thus achieving some lateral cutting or severing as well or instead. Since a few target nerves run along the outer wall region of the vessel along the vessel axis, each at an unknown different clock or angular position, using a trench is a way to statistically intercept and sever more nerves with fewer denervation tool activations. Here are some example trenching approaches for a vessel having a 5 mm interior wall diameter. In Example 1, one forms eight circular-arc trenches oriented along circumferential directions at eight different axial locations. Each trench has a length extending about 45 degrees (to cumulatively cover 360 degrees or more with angle overlap), each trench being offset axially from its neighbor by approximately 1.5 mm. The trench has a trench width of about 0.8 mm (temporary or permanent) and a trench depth of about 4 mm. In Example 2, one forms a trench which is helically situated to wind around 360 degrees or more of the vessel wall and occupying an axial vessel length of a few mm and a pitch angle of 20 degrees. The helical trench has a trench width of about 0.6 mm and a trench depth of about 3.8 mm.

One general preferred technique for trenching with an optical beam is to form an extended trench in incremental length portions, the emitter being moved or redirected only when advancing to the next length portion. This assures that, for example, that a progressive incremental depth-wise severing action is all delivered to the same location into the same trench portion before the optical beam is moved or redirected to the next trench portion.

One general preferred technique for trenching with a penetrating severing tool, whether blade-like or punch-like, is to do a step-and-repeat along the trench length wherein the step length is short enough to assure that the individual severing cuts overlap each other. Tools such as this can utilize snap-action or rapid-actuation and retreat blades or punches which have very short residence times buried in the tissue, thereby minimizing risk of injury.

One general preferred technique for cauterization, particularly thermal cauterization, is to cauterize after the trench or puncture depth has been locally achieved. During the incremental or continuous trenching action associated with this, one can flush the trench using saline or the like to minimize optical losses to any blood within or in front of the trench. However, as mentioned above, also included in the scope is incremental cauterization applied at incrementally achieved trench depths.

One general preferred technique for unheated chemical cauterization is continuous or intermittent flushing of the forming trench such as with a saline/cauterization chemical mixture or a pure cauterization chemical. It will be recognized that for the optical emission severing approach, one can effectively fluidically seal the beam emission/tissue interface with the distal tip, thereby greatly minimizing the flow and total mass of the cauterization chemical employed and thereby easily avoiding any systemic exposure potential issue.

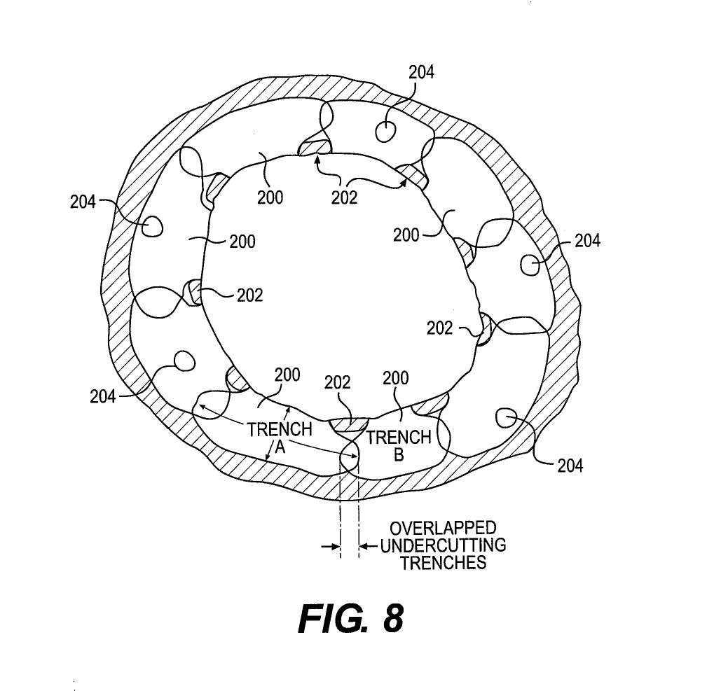

It will be apparent that, for a single blade or puncturing tool passing once along a single severing radial direction into the lumen wall, the maximum length or lateral dimension of tissue severed at the interior wall surface is approximately equal to that severed at the trench bottom. Because it is desirable to intercept as many nerves as possible (or more certainly sever any given nerve), one approach is to create a significant total trench length covering the whole 360 degrees of the lumen wall nerve region at or more preferably somewhat beyond the nominal nerve depth. As discussed above, one can utilize multiple circumferential and separate but angularly overlapping trenches or a helical trench to accomplish this. Less preferred but still within the scope of this invention is the formation of one or more single 360 degree trenches situated substantially on a single circumference of the vessel wall and trenches which are actually strips of trenches (or trench portions) 200 along a trenching path with small webs 202 of connecting tissue left in between them to minimize any possible issue related to trenches later opening up or widening (see FIG. 8). Each bridging web 202 between a pair of adjacent trench portions 200 has connecting tissue left at the interior surface of the vessel wall and is preferably narrower than the pair of adjacent trench portions 200 along the trenching path (generally in the circumferential direction). The trench portions 200 are sufficiently deep in the radial direction to sever the nerves 204. Using a tiltable optical beam, one could easily make a long 360 degree trench with narrow webs 202 bridging the trench portions 200 with tissue separation or removal only at the interior lumen wall but not at nerve-depths.

In some embodiments, the tool is operated to form a plurality of trench portions along a trenching path with webs of connecting tissue left along the path between the trench portions, the remaining webs of connecting tissue being any of: (a) untrenched portions of the lumen wall, (b) portions of the lumen wall which are trenched to a lesser depth than their immediately adjacent trench portions, and (c) portions of the lumen wall which are trenched only beneath the interior lumen surface thereby leaving trench-bridging webs of tissue at the lumen interior surface. In addition, it is possible that the untrenched intervening portions of (a) do not sever nerves, that the shallower trenched intervening portions of (b) do not sever nerves, that the trenching beneath the intervening bridging webs of (c) sever nerves, and that the trench portions themselves sever nerves.

Another enhancement involves making trenches or punctures which have lengths measured at the trench bottom which are greater than the lengths measured at the trench top or vessel interior wall surface. One method to achieve significantly larger at-depth trench length than surface length is to angularly articulate an optical beam (or severing blade) from a given fixed wall position. This forms a trench which gets longer as one goes deeper. The benefit is that, for a given nerve intercept probability (total at-depth trench bottom length), the individual lengths of trenches measured at the vessel wall interior are reduced. This leaves more of the inner vessel wall present to not only reduce wall trauma but to reduce any possibility that stresses, such as blood pressure, perfusion, or temporary stenosis-induced stresses (if any), do not suddenly or gradually split the vessel wall starting at the trench.

As mentioned above, a single helical trench may be formed which wraps around somewhat more than 360 degrees. Its pitch or screw angle relative to a circumferential plane would typically be between about 10 degrees and about 30 degrees with the smaller pitch angles having lower stresses across the trench. The “single” helical trench, upon closer inspection, may actually be a string of separate punctures or trenches (subtrenches or trench portions) each adjacent subtrench pair leaving at least some wall tissue between them to prevent wall tearing. If we also employ the scheme above wherein the optical emission (or mechanical cutting tool) is angularly articulated at each such subtrench, we can physically connect the trenches in the vessel wall subsurface nerve depth region but not at the interior vessel wall surface. This approach provides close to 100% probability of severing all nerves while avoiding any possibility of vessel wall ripping or tearing at trenches or punctures.

Achieving efficacy with the denervation procedure depends on severing at least some nerves and at this time we do not know for sure but suspect that may mean all the nerves surrounding a given vessel. The trenching techniques taught herein allow one to certainly excise all nerves around a 360 degree lumen while minimizing the injury area to the vessel inner wall region in particular. It should be appreciated that prior art placement of multiple separate circular-area lesions via hot ablation damages tissues along the lumen length as well as along the circumference. That damage along the length dimension at the interior vessel wall does not contribute to denervation but does contribute to undesirable reactions such as stenosis or edema, even if temporary. This invention herein achieves maximum effectiveness with minimal undesirable reactions compared to that prior art.

Finally, we note that trenching, whether along circumferences or along helical or other curvilinear paths, can be easily automated as by providing a mechanism to translate the distal tip along the intended trench path. That mechanism may be an angular directing mechanism and an axial translation mechanism, for example. Such automation allows even junior practitioners to still achieve good efficacy and good safety.

Any flushant or irrigant delivered to the distal tip region, whether from the distal portion itself, from another part of the catheter, or from an associated sheath or separate lumen from the catheter, may serve one or both of avoiding optical masking of an optical emitter beam, and cooling the tissue and/or the distal portion such as to further suppress interior vessel wall damage or to prevent fouling or overheating of the optical emission area or coagulator (if used). We have taught the use of chemical cauterization fluid and mention here that that fluid may be delivered separately out of a portion of the catheter or might be delivered, perhaps intermittently, as part of a saline flushant or into a saline flushant path.

In the description, numerous details are set forth for purposes of explanation in order to provide a thorough understanding of the present invention. However, it will be apparent to one skilled in the art that not all of these specific details are required in order to practice the present invention. Additionally, while specific embodiments have been illustrated and described in this specification, those of ordinary skill in the art appreciate that any arrangement that is calculated to achieve the same purpose may be substituted for the specific embodiments disclosed. For example, the tip may also serve as a sensing or pacing electrode or may include a tissue or trench sensor or imaging device. This disclosure is intended to cover any and all adaptations or variations of the present invention, and it is to be understood that the terms used in the following claims should not be construed to limit the invention to the specific embodiments disclosed in the specification. Rather, the scope of the invention is to be determined entirely by the following claims, which are to be construed in accordance with the established doctrines of claim interpretation, along with the full range of equivalents to which such claims are entitled.

What is claimed is:

1. A method for denervation, comprising:
introducing a distal portion of a catheter to an interior of a vessel of a patient, the catheter including an elongated catheter body extending longitudinally between a proximal end and a distal end along a longitudinal axis, the catheter body including the distal portion at the distal end and a catheter lumen from the proximal end to the distal end;
delivering optical energy via the catheter lumen to the distal portion of the catheter body;
emitting an optical beam outwardly from the distal portion, through an optical emission port disposed in the distal portion of the catheter body; and
forming at least one trench using the emitted optical beam with sufficient intensity to a depth into a vessel wall of the vessel sufficient to cause tissue removal and physically sever at least one nerve associated with the vessel wall within a depth range at the depth.

2. The method of claim 1,
wherein the optical beam is emitted to produce severing actions that each sever a portion of the at least one nerve at a time and that cumulatively sever the at least one nerve entirely.

3. The method of claim 1, further comprising:
cauterizing remaining tissue in vicinity of the at least one nerve being physically severed, by directing from the distal portion cauterizing energy to the remaining tissue.

4. The method of claim 3,
wherein the cauterizing is performed by directing an optical beam from the optical emission port to the tissue in vicinity of the at least one severed nerve.

5. The method of claim 3,
wherein forming the at least one trench to physically sever the at least one nerve and the cauterizing of the remaining tissue in the vicinity of the at least one nerve being physically severed are performed substantially sequentially or in a time-interleaved manner.

6. The method of claim 1, further comprising:
flushing blood away from a region adjacent or within the at least one trench with a flushant such as saline emanating from the distal portion.

7. The method of claim 1, further comprising:
physically advancing the optical emission port toward or into the vessel wall before or during forming the at least one trench using the emitted optical beam.

8. The method of claim 1,
wherein the vessel extends longitudinally in a longitudinal direction and the vessel wall is formed circumferentially around the interior of the vessel; and
wherein the at least one trench, when projected longitudinally on a plane normal to the longitudinal direction of the vessel, extends around a complete circumferential loop.

9. The method of claim 8,
wherein the at least one trench is substantially lateral to the longitudinal direction of the vessel.

10. The method of claim 8,
wherein the at least one trench comprises a helical trench.

11. The method of claim 1,
wherein the optical beam used in forming the at least one trench has sufficiently low average power so as to avoid substantially raising a temperature of a region of the vessel wall receiving the emitted optical beam.

12. The method of claim 1,
wherein the optical energy is in an ultraviolet wavelength range.

13. A method for denervation, comprising:
introducing a distal portion of a catheter to an interior of a vessel of a patient, the catheter including an elongated catheter body extending longitudinally between a proximal end and a distal end along a longitudinal axis, the catheter body including the distal portion at the distal end and a catheter lumen from the proximal end to the distal end; and
operating a tool disposed in the distal portion of the catheter to form at least one trench with physical tissue separation in a vessel wall of the vessel and physically sever at least one nerve associated with the vessel using the tool.

14. The method of claim 13,
wherein the physical tissue separation of the at least one trench is temporary.

15. The method of claim 13, further comprising:
cauterizing remaining tissue in vicinity of the at least one nerve being physically severed, by directing from the distal portion cauterizing energy or chemical to the remaining tissue.

16. The method of claim 15, wherein the cauterizing is performed by one of:
(i) directing an optical beam from an optical emission port in the distal portion to the remaining tissue in the vicinity of the at least one nerve being physically severed;
(ii) placing a heated severing tool on the remaining tissue in the vicinity of the at least one nerve being physically severed;
(iii) delivering RF energy to heat the remaining tissue in the vicinity of the at least one nerve being physically severed;
(iv) delivering heat from a heating element in the distal portion toward the remaining tissue in the vicinity of the at least one nerve being physically severed;
(v) emanating a hot fluid from the distal portion toward the remaining tissue in the vicinity of the at least one nerve being physically severed; and
(vi) delivering a chemical cauterizing agent to the remaining tissue in the vicinity of the at least one nerve being physically severed.

17. The method of claim 15,
wherein the physical severing of the at least one nerve and the cauterizing of the remaining tissue in the vicinity of the at least one nerve being physically severed are performed substantially sequentially or in a time-interleaved manner.

18. The method of claim 13,
wherein the physical severing of the at least one nerve is performed by one of (i) delivering optical energy via the catheter lumen to the distal portion of the catheter body, emitting an optical beam outwardly from the distal portion, through an optical emission port disposed in the distal portion of the catheter body, and forming the at least one trench using the emitted optical beam with sufficient intensity to a depth into the vessel wall of the vessel of the patient to cause tissue removal and physically sever the at least one nerve; or (ii) advancing a penetrating severing tool into the vessel wall with physical tissue separation to physically sever the at least one nerve in the vessel.

19. The method of claim 13,
wherein forming the at least one trench is performed without substantially raising a temperature of a region of the vessel wall in vicinity of the at least one trench.

20. The method of claim 19, further comprising:
cauterizing remaining tissue in the vicinity of the at least one nerve being physically severed, by delivering a chemical cauterizing agent to the remaining tissue in the vicinity of the at least one nerve being physically severed without substantially raising a temperature of a region of the vessel wall in the vicinity of the at least one trench.

21. The method of claim 13,
wherein the vessel extends longitudinally in a longitudinal direction and the vessel wall is formed circumferentially around the interior of the vessel; and
wherein the at least one trench, when projected longitudinally on a plane normal to the longitudinal direction of the vessel, extends around a complete circumferential loop.

22. The method of claim 21,
wherein each of the at least one trench has a lateral length which is greater than a width and a depth into the vessel wall which is greater than the width, the lateral length being substantially perpendicular to the longitudinal direction of the vessel.

23. The method of claim 22,
wherein each of the at least one trench has a surface length along an interior surface of the vessel wall and an at-depth length at a trench bottom of the trench at the depth into the vessel wall, the at-depth length being greater than the surface length of the trench.

24. The method of claim 21,
wherein the at least one trench comprises a helical trench.

25. The method of claim 13,
wherein the tool comprises a pair of diametrically opposed penetrating severing tools; and
wherein the penetrating severing tools are operated to cut simultaneously dual diametrically opposed trenches at least partially through the vessel wall.

26. The method of claim 13,
wherein the tool comprises a penetrating severing tool; and
wherein operating the tool comprises translating the penetrating severing tool while advancing the penetrating severing tool into the vessel wall to form the at least one trench in the vessel wall.

27. The method of claim 26,
wherein operating the tool comprises translating on surface of the vessel wall, cutting, translating on surface, cutting, and translating on surface.

28. The method of claim 26,
wherein operating the tool comprises cutting and remaining penetrated, translating while penetrated, and sideways cutting relative to the translating.

29. The method of claim 13,
wherein the tool comprises optical energy delivered via the catheter lumen to the distal portion of the catheter body, to emit an optical beam outwardly from the distal portion, through an optical emission port disposed in the distal portion of the catheter body, so as to form the at least one trench using the emitted optical beam with sufficient intensity to a depth into the vessel wall of the vessel of the patient to cause tissue removal and physically sever the at least one nerve; and
wherein operating the tool includes maintaining an optical path for the emitted optical beam to a trench bottom of the at least one trench.

30. The method of claim 13, further comprising:
directing a fluid from the distal portion to flush the at least one trench.

31. The method of claim 13, wherein the tool is operated to form a plurality of trench portions along a trenching path with webs of connecting tissue left between the trench portions along the trenching path, the webs of connecting tissue being any of:
(a) untrenched portions of the lumen wall,
(b) portions of the lumen wall which are trenched to a lesser depth than their immediately adjacent trench portions, or
(c) portions of the lumen wall which are trenched only beneath the interior lumen surface thereby leaving bridging webs of tissue at the lumen interior surface.

32. The method of claim 31,
wherein the webs of connecting tissue do not reach a depth of the at least one nerve to be physically severed.
